(19) United States
(12) Patent Application Publication
(10) Pub. No.: US 2026/0274925 A1
(43) Pub. Date: Sep. 17, 2026

(54) PROCESS FOR THE PRODUCTION OF RECOMBINANT PROTEINS

(71) Applicant: UCB BIOPHARMA SRL, BRUSSELS (BE)

(72) Inventors: GEOFFREY NORMAN BROWN, SLOUGH, BERKSHIRE (GB); RICHARD BARRY DAVIES, SLOUGH, BERKSHIRE (GB)

(21) Appl. No.: 19/119,843

(22) PCT Filed: Oct. 10, 2023
(86) PCT No.: PCT/EP2023/078040

§ 371 Date: Apr. 10, 2025

(30) Foreign Application Priority Data
Oct. 11, 2022 (GB) 2214946.2
Jun. 7, 2023 (GB) 2308473.4

(51) Int. Cl. C07K 16/00; C07K 1/14

(52) U.S. Cl. CPC C07K 16/00; C07K 1/14; C07K 2317/14; C07K 2317/55

(57) ABSTRACT

The invention relates to the field of recombinant production of proteins in host cells. In particular, the invention relates to processes for manufacturing recombinant proteins in host cells which processes reduce the misincorporation of norleucine in place of methionine.

### FIELD OF THE INVENTION

The invention relates to the field of recombinant production of proteins in host cells. In particular, the invention relates to processes for culturing host cells for the production of recombinant proteins in order to reduce the misincorporation of norleucine in place of methionine.

### BACKGROUND OF THE INVENTION

In the field of medicine, the use of biological entities, such as proteins, e.g. antibodies or antibody-derived molecules, has been constantly gaining presence and importance. With it, the need for controlled manufacturing processes has developed. The commercialization of proteins for medical use requires that they be produced in large amounts, and a lot of effort has been dedicated to improving the culturing of recombinant host cells that express the desired protein and their processing. This has resulted in increased product titres, but often also a higher amount of undesired by-products and increased product heterogeneity are observed. As the removal of such undesired by-products or product variants may be laborious, it would be preferable to optimise the manufacturing processes such that their formation is minimised.

One undesirable product variation results from the misincorporation of norleucine into proteins instead of methionine. Norleucine is an unnatural amino acid synthesized by the enzymes of the leucine biosynthetic pathway in E. coli.It is a structural analogue of methionine and can substitute for methionine residues in proteins because methionyl-tRNA synthetase (MetRS) can use norleucine as a substrate, albeit at a lower efficiency when compared to methionine, to charge the methionyl-tRNA during the translation process.

It is known since the 1950's that when expressed in E. coli many heterologous proteins have norleucine mistakenly incorporated in places methionine residues should appear (Munier and Cohen 1956 and Nisman and Hirsch 1958). The misincorporation of norleucine is undesirable because it results in the production of an altered protein, i.e. a protein with different primary amino acid sequence, with potentially unknown characteristics. It has been shown that the misincorporation of a non-natural amino acid can alter the 3D structure of a protein and result in aggregation. Norleucine misincorporation occurs to various degrees in manufacturing batches and thus leads to heterogeneity of batches of product.

Even though norleucine misincorporation can be reduced by the increase in the concentration of methionine in the cell culture medium (Tsai at al., Biochem Biophys Res Comm 156:733, 1988, Bogosian et al., J Biol Chem 264:531, 1989, U.S. Pat. No. 5,599,690 and WO 2007/103521) this has various disadvantages, including increasing the operational complexity and the cost of manufacture (Veeravalli and Laird, Bioengineered 6:132, 2015). Additional methods have been developed to reduce norleucine incorporation in recombinant proteins, such as expressing norleucine degrading enzymes (U.S. Pat. No. 8,603,781) or deleting genes involved in biosynthesis of norleucine (Bogosian et al., J Biol Chem 264:531, 1989).

However, genetic modifications of host cell strains are cumbersome and may have other unexpected or unidentified effects. Altering cell culture media may lead to increased formation of other undesired by-products and/or have other negative effects on cell culture performance. Thus, there is still a need for new processes to prevent or reduce misincorporation of norleucine in proteins during manufacture. This need is addressed by the present invention.

### SUMMARY OF THE INVENTION

In a first embodiment, the invention relates to a process for producing a recombinant protein comprising the steps of:
a) providing host cells capable of producing a recombinant protein,
b) providing an amount of liquid medium containing between 0 and 1 g methionine per kg of liquid medium,
c) culturing said host cells in the liquid medium,
d) inducing production of the recombinant protein in the liquid medium, and
e) at the time or after induction adding an amount of methionine to the liquid medium while culturing said host cells to produce the recombinant protein
   wherein the amount of methionine per kg of liquid medium added in step (e) is higher than the amount of methionine per kg of liquid medium contained in the liquid medium in step (b).

In a second embodiment, the invention relates to a process for reducing norleucine misincorporation during the production a recombinant protein comprising the steps of:
a) providing host cells capable of producing a recombinant protein,
b) providing an amount of liquid medium containing between 0 and 1 g methionine per kg of liquid medium,
c) culturing said host cells in the liquid medium,
d) inducing production of the recombinant protein in the liquid medium, and
e) at the time or after induction adding an amount of methionine to the liquid medium while culturing said host cells to produce the recombinant protein
   wherein the amount of methionine per kg of liquid medium added in step (e) is higher than the amount of methionine per kg of liquid medium contained in the liquid medium in step (b).

In an even further embodiment, the invention relates to a recombinant protein preparation obtainable or obtained by the process according to any one of the preceding claims.

### BRIEF DESCRIPTION OF THE DRAWINGS

FIG. 1 shows the growth profiles of various batches as determined by the optical density at 600 nm (OD600) of E. coli during fermentation in 200 L vessels as described in Example 1 with no methionine added, methionine added before induction [step (c)] and at or after induction [step (e)] or only at or after induction [step (e)].

FIG. 2 shows the cell viability of E. coli in various batches of fermentation in 200 L vessels as described in Example 1 with no methionine, methionine added before induction [step (c)] and at or after induction [step (e)] or only at or after induction [step (e)].

FIG. 3 shows the titre of Fab′ in various batches after harvest of E. coli fermentation in 200 L vessels as described in Example 1 with no methionine, methionine added before induction [step (c)] and at or after induction [step (e)] or only at or after induction [step (e)].

FIG. 4 shows the average norleucine level per methionine residue in a Fab′ in produced in batches of E. coli fermentation in 200 L vessels as described in Example 1 with no methionine added, methionine added before induction [step (c)] and at or after induction [step (e)] or only at or after induction [step (e)].

FIG. 5 shows the growth profiles as determined by the OD600 in various batches of E. coli fermentation in 15,000 L vessels as described in Example 2 with no methionine added, methionine added before induction [step (c)] and at or after induction [step (e)] or only at or after induction [step (e)].

FIG. 6 shows the average norleucine level per methionine residue in a Fab′ produced in batches of E. coli fermentation in 15,000 L vessels as described in Example 2 with no methionine added, methionine added before induction [step (c)] and at or after induction [step (e)] or only at or after induction [step (e)].

FIG. 7 shows the average norleucine level per methionine residue for 3 different manufacturing processes yielding different amounts of product (Process A, B and C) all operated at the same scale to produce the same Fab′. Process A is a low yielding process with no methionine added to the feed, whereas processes B and C are improved higher yielding process. Process B does not involve addition of methionine to the feed whereas Process C is carried out according to the invention.

FIG. 8 shows the average norleucine level per methionine residue for Processes A and C.

### DETAILED DESCRIPTION OF THE INVENTION

It has surprisingly been found by the inventors of the instant invention that methionine addition to the cell culture medium during the growth and expansion phase of the cell culture decreases or inhibits cell growth or expansion.

Based on this surprising finding, the inventors have devised new and improved manufacturing processes which overcome the problems associated with the processes of reducing norleucine misincorporation during the manufacture of proteins known in the art.

As used herein, the singular forms “a”, “an” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, include the phrases “consisting essentially of”, “consists essentially of”, “consisting”, and “consists” can be used interchangeably. The phrases “consisting essentially of” or “consists essentially of” indicate that the claim encompasses embodiments containing the specified materials or steps and those that do not materially affect the basic and novel characteristic(s) of the claim.

The present invention relates to processes of culturing cells for the production of recombinant proteins. In the process of the invention, methionine is added to the medium in order to minimise norleucine misincorporation in a particular way such that negative effects of methionine on cell growth are minimised.

Thus, in a first embodiment, the invention relates to a process for producing a recombinant protein comprising the steps of:
a) providing host cells capable of producing a recombinant protein,
b) providing an amount of liquid medium containing between 0 and 1 g methionine per kg of liquid medium,
c) culturing said host cells in the liquid medium,
d) inducing production of the recombinant protein in the liquid medium, and
e) at the time or after induction adding an amount of methionine to the liquid medium while culturing said host cells to produce the recombinant protein
   wherein the amount of methionine per kg of liquid medium added in step (e) is higher than the amount of methionine per kg of liquid medium contained in the liquid medium in step (b).

In a second embodiment, the invention relates to a process for reducing norleucine misincorporation during the production a recombinant protein comprising the steps of:
a) providing host cells capable of producing a recombinant protein,
b) providing an amount of liquid medium containing between 0 and 1 g methionine per kg of liquid medium,
c) culturing said host cells in the liquid medium,
d) inducing production of the recombinant protein in the liquid medium, and
e) at the time or after induction adding an amount of methionine to the liquid medium while culturing said host cells to produce the recombinant protein
   wherein the amount of methionine per kg of liquid medium added in step (e) is higher than the amount of methionine per kg of liquid medium contained in the liquid medium in step (b).

In step (a) host cells are provided which are capable of producing a recombinant protein upon induction. The host cells being used in the process of the invention can be any host cell which is suitable for the recombinant production of proteins and able to grow under the specified conditions. Suitable host cells include bacterial host cells and other cells which can exhibit norleucine misincorporation in place of methionine.

In a third embodiment, the host cell in the process according to any one of the first, second or any other embodiment of the invention is a bacterial host cell, such as E. coli cell or another gram-negative bacterial cell or a gram-positive bacterial cell, such as e.g. Staphylococcus aureus.In a more preferred embodiment according to the third embodiment, the host cell is an E. coli host cell, even more preferred of strain HB101, B7, K12, RV308, DH1, HMS174, W3110 or BL21.

Typically, a nucleic acid sequence encoding the recombinant protein under the control of an inducible promoter has been introduced into the host cell. Suitable vectors for expressing such nucleic acid constructs in host cells and processes for transformation of host cells are well-known in the art. Suitable inducible promoters are also well-known in the art and some, non-limiting, examples are mentioned herein below.

In step (c) of the processes according to any one of the embodiments of the invention as described herein said host cells are cultured. Processes and media for the culturing of various types of host cells are well-known in the art. Media vary according to the organism, but may comprise components such as a carbon source, a nitrogen source, amino acids, vitamins, essential metal ions and trace elements, etc. Step (c) preferably includes fed-batch culturing, more preferably in a bioreactor. The fed-batch phase may be preceded by a batch phase. Inoculation may occur directly from a working cell bank or via a seed culture, e.g. in a shake flask.

In a fourth embodiment of the invention, step (c) in the process according to any one of the first, second, third or any other embodiment of the invention comprises growing the culture to an OD600 (optical density at a wavelength of 600 nm) of at least 20, such as at least 25, at least 35, at least 50, at least 55, at least 60, at least 70, or at least 80.

In a fifth embodiment, the liquid medium in step (b) and/or step (c) of the process according to any one of the first, second, third, fourth or any other embodiment of the invention contains less than 1 g methionine per kg of liquid medium, e.g. less than 1 g/kg, such as less than 0.5 g/kg, less than 0.25 g/kg, less than 0.20 g/kg, less than 0.15 g/kg, less than 0.10 g/kg, in each case per kg of liquid medium. Preferably, the concentration of methionine in the liquid medium in steps (b) and/or (c) is between 0 and 0.25 g/kg or between 0 and 0.5 g/kg.

In a sixth embodiment, no methionine is contained in the liquid medium in step (b) and/or step (c), that means prior to induction, of the process according to any one of the first, second third, fourth, fifth or any other embodiment of the invention.

In a seventh embodiment, no isoleucine is contained in the liquid medium of step (b) and/or step (c), that means prior to induction, of the process according to any one of the first, second third, fourth, fifth, sixth or any other embodiment of the invention.

In an eighth embodiment, no leucine is contained in the liquid medium of step (b) and/or step (c), that means prior to induction, of the process according to any one of the first, second third, fourth, fifth, sixth, seventh or any other embodiment of the invention.

In a ninth embodiment, step (c) of the process according to any one of the first, second third, fourth, fifth, sixth, seventh, eighth or any other embodiment of the invention comprises:
i) culturing said host cells in batch culture to an OD600 of between 20 and 55, wherein optionally a bolus amount (i.e. a single dose added at one time) of a magnesium salt, such as magnesium sulphate is added,
ii) further culturing said bacterial host cells whereby the OD increases until the dissolved oxygen (DO) increases to ≥50% of air saturation, and
iii) culturing said host cells in fed-batch culture until the OD600 in the liquid medium of the culture increases by at least 15, 20, 25, 35, 40 or 50 units above the OD600 in step (c)(i).

In a tenth embodiment, a feed containing a carbon source is added starting in step (c)(iii) of the process of the invention according to the ninth embodiment. Preferably, the amount of carbon source added to the liquid medium per unit time is lower in step (e) than during step (c)(iii), e.g. by lowering the feed rate or by reducing the concentration of the carbon source in the feed. In a preferred embodiment, no methionine is present in the liquid medium or added to the liquid medium prior to the addition of the feed with a carbon source according to the tenth embodiment of the invention.

In an eleventh embodiment, the induction of the production of the recombinant protein according to step (d) of the process according to any one of the first, second third, fourth, fifth, sixth, seventh, eighth, ninth, tenth or any other embodiment of the invention is initiated when the dissolved oxygen (DO) increases to 50% of air saturation in the liquid medium of culture or when a predefined OD600 is reached as defined in step (c)(iii) of the ninth embodiment. DO may be measured by any standard means such as an online polarographic dissolved oxygen sensor, an optical dissolved oxygen sensor or any other appropriate oxygen sensing technology.

In a preferred embodiment, in the process according to any one of the embodiments of the invention there is no production, or production of less than 0.1 g of the recombinant protein per kg of liquid medium prior to the induction according to step (d).

Induction of production of the recombinant protein can be achieved by any suitable method. In one embodiment, in the process according to any one of the embodiments of the invention a gene encoding the recombinant protein is under the control of an inducible promoter. Inducible promoters are known in the art. A well-known bacterial expression system using an inducible promoter, is a system wherein the gene encoding the recombinant protein is placed under the control of a lac-type promoter, which can be induced by IPTG (Isopropyl β-D-I-thiogalactopyranoside). Other known bacterial expression systems include e.g. the arabinose promoter system (see e.g. Guzman et al., J Bacteriol 177:4121, 1995) or the T7 system (see e.g. Rosenberg et al., Gene 56:125, 1987). These and other systems have been reviewed, e.g. in Rosano and Ceccarelli, Front Microbiol 5:172, 2014.

In a preferred embodiment of the process of the invention, the host cells in the process according to any one of the embodiments of the invention comprise a nucleic acid sequence encoding the recombinant protein under the control of an IPTG-inducible promoter and thus produce recombinant protein upon induction with IPTG. In such an embodiment, step (d) comprises addition of IPTG.

Step (e) in the process according to any one of the embodiments of the invention typically includes fed-batch culturing in a bioreactor. In one embodiment, the duration of step (e) of the process according to any of the embodiments of the invention is between about 12 and about 96 hours, such as between about 20 and about 72 hours, e.g. between about 24 and about 48 hours or between about 25 and about 55 hours, such as between 30 and about 50 hours or between 35 and 45 or between 36 and 48 hours. In the context of time, the term “about” is intended to include ±1, ±2, ±3, ±4, ±5, ±6, ±7, ±8, ±9, or ±10 hour(s).

In a twelfth embodiment of the invention of the process according to any one of the first, second third, fourth, fifth, sixth, seventh, eighth, ninth, tenth, eleventh or any other embodiment of the invention step (e) is followed by a step (f) which is harvesting the host cells. Preferably the amount of methionine added in step (e) is such that the concentration of methionine in the liquid medium at or immediately prior to harvest in step (f) is at least 0.25 g/kg, such as between 0.25 g/kg and 1.5 g/k and preferably at least 0.40 g/kg, such as between 0.40 g/kg and 1.2 g/kg. More preferably, the amount added in step (e) is such that the concentration of methionine in the liquid medium at or immediately prior to harvest in step (f) is between 0.45 g/kg and 1.10 g/kg, e.g. between 0.50 g/kg and 0.9 g/kg.

In a thirteenth embodiment of the invention the amount of methionine added in step (e) of the process according to any one of the first, second third, fourth, fifth, sixth, seventh, eighth, ninth, tenth, eleventh, twelfth or any other embodiment of the invention is at least 0.25 g/kg of liquid medium provided in step (b), such as between 0.25 g/kg and 2.0 g/k and preferably at least 0.50 g/kg, such as between 0.50 g/kg and 1.2 g/kg of liquid medium provided in step (b). More preferably, the amount is between 0.52 g and 1.10 g per kg of liquid medium in step (b), e.g. between 0.55 g and 1.05 g per kg of liquid medium provided in step (b).

In a fourteenth embodiment, no leucine is added during step (e) of the process according to any one of the first, second third, fourth, fifth, sixth, seventh, eighth, ninth, tenth, eleventh, twelfth, thirteenth or any other embodiment of the invention.

In a fifteenth embodiment, no isoleucine is added during step (e) of the process according to any one of the first, second third, fourth, fifth, sixth, seventh, eighth, ninth, tenth, eleventh, twelfth, thirteenth, fourteenth or any other embodiment of the invention.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. When ranges are used herein, specific embodiments of different combinations and sub-combinations of these ranges (e.g., subranges within the disclosed ranges) are intended to be explicitly included.

### Carbon Source

The process of the invention typically includes the addition of one or more organic carbon sources. The carbon source used may be a single type of carbon source or a mixture of different carbon sources. Suitable carbon sources include e.g. glucose, lactose, arabinose, glycerol, sorbitol, galactose, xylose or mannose. As an example, more than 75%, e.g. at least 90%, of the carbon source in the liquid medium in step (b) consists of glycerol. In another preferred embodiment, more than 75%, e.g. at least 90%, of the carbon source in the liquid medium in step (e) of the process of the invention consists of glycerol. As another example, more than 75%, e.g. at least 90%, of the carbon source in the liquid medium in step (c) consists of glucose. In another preferred embodiment, more than 75%, e.g. at least 90%, of the carbon source in the liquid medium in step (e) consists of glucose. As a further example, more than 75%, e.g. at least 90%, of the carbon source in the liquid medium in step (c) consists of lactose. In another preferred embodiment, more than 75%, e.g. at least 90%, of the carbon source in the liquid medium in step (e) consists of lactose.

### pH

The pH of the cell culture medium during fermentation is very important for product yield and processability of the cell culture slurry. The formation of struvite has been described to be influenced by pH (see e.g. Pérez-García et al., 1989). In an embodiment of the process of the invention, the pH of the culture in step (c) of the process according to any of the embodiments of the invention is above 6.5, such as 6.6, 6.7, 6.8, 6.9, 7.0, 7.1, 7.2 or is above about 6.5, such as about 6.6, about 6.7, about 6.8, about 6.9, about 7.0, about 7.1, or about 7.2 and the pH of the culture in step (e) is above 6.5, such as 6.6, 6.7, 6.8, 6.9, 7.0, 7.1, 7.2 or is above about 6.5, such as about 6.6, about 6.7, about 6.8, about 6.9, about 7.0, about 7.1, or about 7.2. In another embodiment, the pH in step (c) is between 6 and 8, such as between 6.5 and 7.5, e.g. between 6.6 and 7.4, such as between 6.7 and 7.3, e.g. between 6.8 and 7.2 and the pH in step (e) is between 6 and 8, such as between 6.5 and 7.5, e.g. between 6.6 and 7.4, such as between 6.7 and 7.3, e.g. between 6.8 and 7.2. In another embodiment, the pH in step (c) is between about 6 and about 8, such as between about 6.5 and about 7.5, e.g. between about 6.6 and about 7.4, such as between about 6.7 and about 7.3, e.g. between about 6.8 and about 7.2 and the pH in step (e) is between about 6 and about 8, such as between about 6.5 and about 7.5, e.g. between about 6.6 and about 7.4, such as between about 6.7 and about 7.3, e.g. between about 6.8 and about 7.2. In the context of pH, the term “about” is intended to include ±0.1, ±0.2, or ±0.3 pH unit.

### Temperature

The temperature is typically kept as constant as possible throughout the full fermentation process. In certain embodiments, the temperature is maintained at a constant temperature of 25° C., 26° C., 27° C., 28° C., 29° C., 30° C., 31° C., 32° C., 33° C., 34° C., or 35° C. In other embodiments, the temperature can be maintained at a temperature of a constant temperature of about 25° C., about 26° C., about 27° C., about 28° C., about 29° C., about 30° C., about 31° C., about 32° C., about 33° C., about 34° C., or about 35° C. In the context of temperature, the term “about” is intended to include ±1° C., ±2° C., or ±3 ° C. or a set temperature (e.g., a range of ±0 ° C. to 3° C. around a set temperature).

### Recombinant Proteins

The recombinant protein produced in the process of the invention is typically a heterologous protein, originating from another organism. For example, the recombinant protein may be an antibody, cytokine, growth factor, hormone or other peptide or polypeptide or a derivative of fusion protein of any of the foregoing.

In a preferred embodiment, the recombinant protein is an antibody. The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG₁, IgG₂ₐ, IgG_{2b}, IgG₃, IgG₄, IgE, IgD and antibodies that are produced as dimers of this basic structure including IgGA₁, IgGA₂, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe) or Fab-(dsscFv)₂ (also referred to as TrYbe, see WO2015/197772 for instance). Antibody fragments as defined above are known in the art. In a preferred embodiment, the recombinant protein produced is a Fab or Fab′ fragment. In a further preferred embodiment, the recombinant protein is certolizumab pegol, dapirolizumab pegol, ranibizumab, abciximab, blinatumomab, idarucizumab, moxetumomab pasudotox, caplacizumab, brolucizumab.

The process according to any one of the embodiments of the invention can in principle take place in any suitable container such as a shake flask or a bioreactor, which may or may not be operated in a fed-batch mode depending e.g. on the scale of production required.

In a sixteenth embodiment, at least steps (c), (d) and (e) of the process according to any one of the first, second third, fourth, fifth, sixth, seventh, eighth, ninth, tenth, eleventh, twelfth, thirteenth, fourteenth, fifteenth or any other embodiment of the invention are performed in a bioreactor, preferably an industrial scale bioreactor. The bioreactor may e.g. be a stirred-tank or air-lift reactor. The bioreactor maybe a reusable reactor made of glass or metal, e.g. stainless steel, or a single-use bioreactor made of synthetic material, such as plastic.

In a seventeenth embodiment, at least step (e) of the process according to any one of the first, second third, fourth, fifth, sixth, seventh, eighth, ninth, tenth, eleventh, twelfth, thirteenth, fourteenth, fifteenth, sixteenth or any other embodiment of the invention is carried out in a bioreactor with a volume of equal or more than 100 L, equal or more than 500 L, equal or more than 1,000 L, equal or more than 2,000 L, equal or more than 5,000 L, equal or more than 10,000 L or equal or more than 20,000 L, 1,000 to 30,000 L, 5,000 to 30,000 L, 10,000 to 30,000 L, 1,000 to 20,000 L, 5,000 to 20,000 L, 10,000 to 20,000 L or 10,000 to 25,000 L.

In an eighteenth embodiment, in step (b), (c), (d) or (e) of the process according to any one of the first, second third, fourth, fifth, sixth, seventh, eighth, ninth, tenth, eleventh, twelfth, thirteenth, fourteenth, fifteenth, sixteenth, seventeenth or any other embodiment of the invention the liquid medium of the culture has a volume of equal or more than 100 L, equal or more than 500 L, equal or more than 1,000 L, equal or more than 2,000 L, equal or more than 5,000 L, equal or more than 10,000 L or equal or more than 20,000 L, 1,000 to 30,000 L, 5,000 to 30,000 L, 10,000 to 30,000 L, 1,000 to 20,000 L, 5,000 to 20,000 L, 10,000 to 20,000 L or 10,000 to 25,000 L. In a further preferred embodiment of the process according to any of the embodiments of the invention, in all of steps (b), (c), (d) and (e) the culture has a volume of equal or more than 100 L, equal or more than 500 L, equal or more than 1,000 L, equal or more than 2,000 L, equal or more than 5,000 L, equal or more than 10,000 L or equal or more than 20,000 L, 1,000 to 30,000 L, 5,000 to 30,000 L, 10,000 to 30,000 L, 1,000 to 20,000 L, 5,000 to 20,000 L, 10,000 to 20,000 L or 10,000 to 25,000 L.

The process according to any one of the embodiments of the invention may comprise one or more further steps after step (e). For instance, the process may comprise the further step of recovering the recombinant protein, which may comprise first separating cells from supernatant or from inclusion bodies. Once recovered, the recombinant protein can be isolated and purified. Isolation and purification processes are well-known to those skilled in the art. They typically consist of a combination of various chromatographic and filtration steps. The process of the invention may further comprise the step of formulating the recombinant protein into a pharmaceutical composition suitable for medical use, e.g. therapeutic or prophylactic use. In one embodiment, the recombinant protein is modified, such as conjugated to another molecule, before being formulated into a pharmaceutical composition.

In a nineteenth embodiment the process according to any one of the first, second third, fourth, fifth, sixth, seventh, eighth, ninth, tenth, eleventh, twelfth, thirteenth, fourteenth, fifteenth, sixteenth, seventeenth, eighteenth or any other embodiment of the invention comprises lyophilizing the composition comprising the recombinant antibody produced according to any of the embodiments of the process of the invention.

A further embodiment of the invention is a recombinant protein preparation, such as an antibody preparation, preferably a preparation comprising certolizumab pegol, dapirolizumab pegol, ranibizumab, abciximab, blinatumomab, idarucizumab, moxetumomab pasudotox, caplacizumab, brolucizumab, obtained or obtainable according to the process according to any one of the first, second third, fourth, fifth, sixth, seventh, eighth, ninth, tenth, eleventh, twelfth, thirteenth, fourteenth, fifteenth, sixteenth, seventeenth, eighteenth, nineteenth or any other embodiment of the invention.

### Detection and Quantification of Norleucine Misincorporation

Methods for the detection of norleucin misincorporation are known in the art and reviewed in Steele et al., Proteomes 9(1):2, 2021. A preferred method for analysis and quantification of norleucin misincorporation is mass spectrometry.

### EXAMPLES

Example 1

A frozen cell bank vial containing E. coli W3110 host cells expressing Antibody A (a Fab′ fragment having a pl in the range 8.8-9.3.) was used to inoculate a shake flask containing 6× peptone-yeast extract (6×P—Y) medium plus tetracycline. This shake flask was incubated at 30° C. and 200-250 rpm. At the required OD range, the shake flask was used to inoculate a seed fermenter containing a chemical defined medium (derived from the MD media from Durany et al. 2004) plus tetracycline, with a carbon source. The cell culture within the seed fermenter was maintained at 30° C. At the required OD range, the seed culture was used to inoculate the production fermenter (175 kg liquid medium) containing the same chemically defined medium as used in the seed fermenter. The production fermenter was maintained in the same conditions as the seed fermenter and grown in the batch phase until the carbon source was depleted. During this time a bolus addition of MgSO₄ was made to avoid depletion of this metabolite. At the end of the batch phase (signalled by a spike in the measured DO), an exponential carbon source feed [containing various amounts of methionine corresponding to between 0 and 0.70 g per kg of liquid medium provided in step (b) depending on the batch] was switched on and the culture was fed with a specific amount of carbon source to achieve an OD600 of greater than 50 units. At this point, the carbon source feed was switched from an exponential phase feed to a production phase feed [containing various amounts of methionine corresponding to between 0.50 g and 1.5 g per kg of liquid medium provided in step (b)] and the Antibody A expression was induced by the addition of IPTG. Cells (containing the expressed Antibody A) were harvested after more than 40 hours post induction.

Cells were harvested by continuous centrifugation. Concentrated cell slurry was resuspended back to the original cell harvest concentration by the addition of deionised water and concentrated Tris EDTA extraction buffer to achieve the required buffer concentration. For heat extraction, the cells maintained at an elevated temperature with mixing for a defined period of time.

Fab′ Concentration: Harvest Fab′ concentrations were determined using Protein G HPLC analysis in 20 mM Phosphate Buffer. Elution was via a pH gradient from pH 7.4 on injection, reducing to pH 2.7 Protein L Purification: Samples of extract were purified by Protein L affinity chromatography using 600 μL Capto L® columns in order to purify the cell extract prior to analysis of norleucine misincorporation levels. Columns were prepared by flushing with a Phosphate/Sodium Chloride buffer (Buffer A), cleaning with a Sodium Hydroxide solution and then an equilibrium step with buffer A. After sample loading a wash was performed with Buffer A before elution using a Glycine buffer. A fraction of the eluate was collected as appropriate to recover a representative Fab′ sample for norleucine misincorporation analysis.

Analysis of norleucine misincorporation levels: To perform the analysis the sample was enzymatically digested with trypsin into fragment peptides. These were subsequently separated using liquid chromatography prior to online analysis using electrospray ionisation with mass spectrometry. Mass spectrometry measures the mass-to-charge ratio of the peptides, from which their mass may be inferred. A peptides mass is a highly specific characteristic of the peptide's sequence. The retention time and mass of each of the peptides observed is unique to the amino acid sequence of the peptide, which allows comparison of the masses and retention times of the peptides observed to those of the theoretical sequence. The high sensitivity of mass spectrometry allows low levels of protein modifications to be detected.

The substitution of a methionine residue with a norleucine residue will reduce the mass of a peptide by 17.9564 Da. This mass shift combined with tandem mass spectroscopy (MSMS fragmentation) allows peptides that contain a norleucine substitution to be confirmed. A semi-quantitative assessment of the levels of norleucine misincorporation may be determined by measurement of the peak area of the extracted ion chromatograms (EIC) of the norleucine and methionine containing peptides.

A peptide mapping method that uses liquid chromatography (LC) and mass spectrometry (MS) with an Orbitrap Q-Exactive plus mass spectrometer was for the semi-quantitative determination of the levels of norleucine misincorporation in protein samples.

Cell Viability Measurement: Cell viability was monitored using a FACSCalibur flow cytometer.

The cells were first stained with BOX and PI dyes.

DO measurement: Dissolve oxygen (DO) was measured using an online polarographic dissolved oxygen sensor.

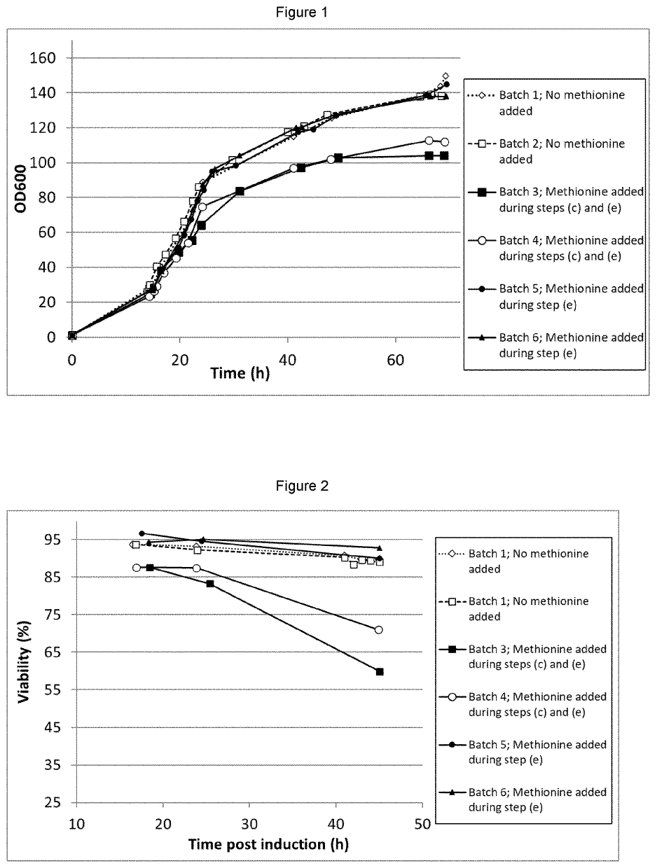

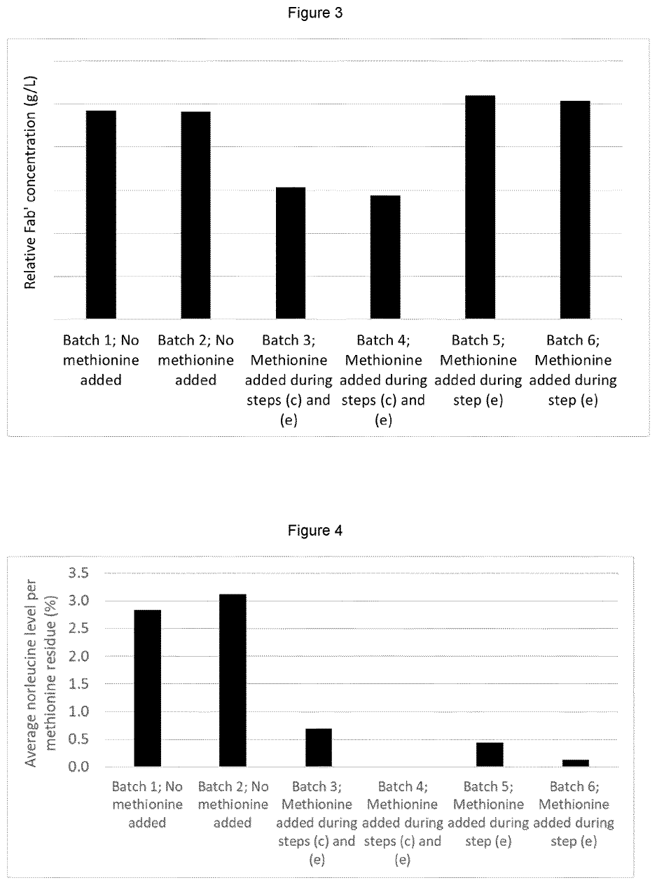

In this example fermentations were carried out with no addition of methionine at all, methionine included in the feed used prior to induction and at or after induction [i.e. in steps (c) and (e) of the process] and methionine included in only the feed at or after induction [i.e. in step (e)] of the process. When methionine was included in the feed used in steps (c) and (e) the cell growth (FIG. 1), the cell viability (FIG. 2) and the Fab′ concentration (FIG. 3) were all reduced compared to when methionine was not added to the process or only added in the feed use in step (e). It can be seen from the same Figures that there is no significant difference in these parameters (cell growth, viability and titre) between fermentations grown without methionine and those grown with methionine in the feed used in step (e). FIG. 4 shows that adding methionine to the feed in step (e) was sufficient to reduce the average level of norleucine misincorporation per methionine residue (while not impacting the other process parameters as mentioned above).

### Example 2

The fermentations were carried out providing approximately 10,000 kg of liquid media for step (b). The process described in Example 1 was scaled as appropriate to adjust for the increased starting volume. All scale independent parameters (e.g. temperature, pH, DO set point) were kept the same as in Example 1.

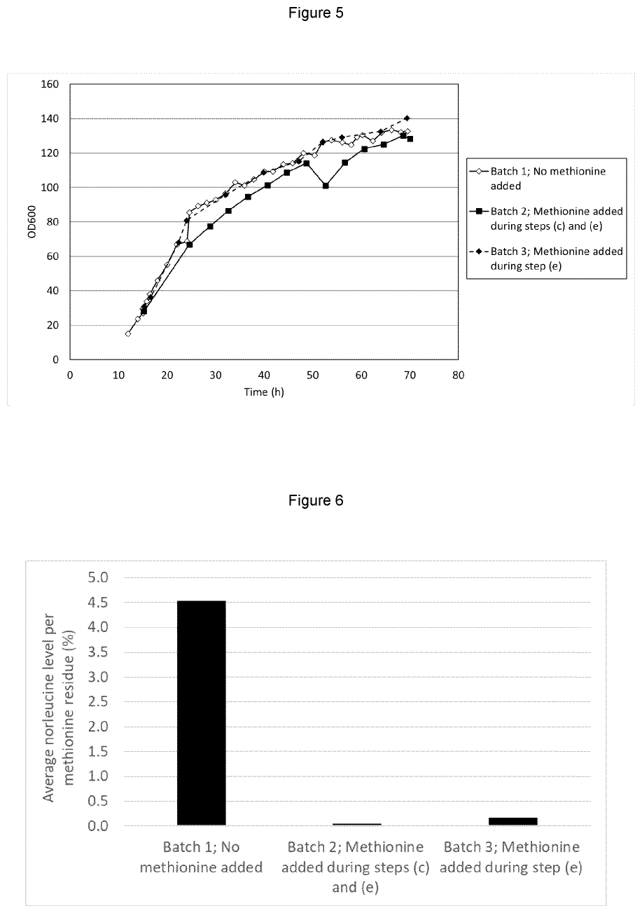

It can be seen from FIG. 5 that adding methionine to the feed prior to induction and at or after induction [i.e. Steps (c) and (e)] caused the cell growth to be reduced, whilst adding methionine only to the feed used at or after induction [i.e. in step (e)] did not impact the growth compared to when no methionine was added. FIG. 6 shows that adding methionine to the feed in step (e) was sufficient to reduce the average level of norleucine misincorporation.

### Example 3

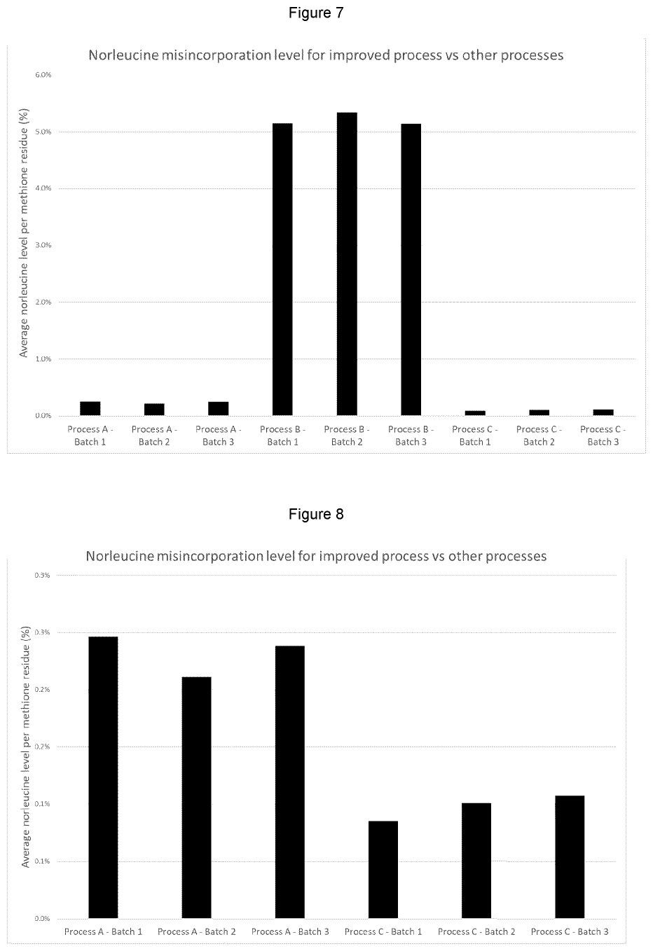

FIG. 7 shows the average level of norleucine misincorporation per methionine residue for 3 representative batches of 3 processes all run with approximately 10,000 kg of liquid media provided in step (b). Process A is a lower yielding fermentation process with no addition of methionine to the feed. Process B was developed as a higher yielding process with no addition of methionine to the feed to make the same Fab′, and as can be seen from FIG. 7 resulting in much higher norleucine misincorporation compared to process A. Process C was a further development of Process B which included the application of this invention and resulted in a lower norleucine misincorporation level than the original process (see FIG. 8).

1-20. (canceled)

21. A process for producing a recombinant protein comprising the steps of:
a) providing host cells capable of producing a recombinant protein,
b) providing an amount of liquid medium containing between 0 and 1 g methionine per kg of liquid medium,
c) culturing said host cells in the liquid medium,
d) inducing production of the recombinant protein in the liquid medium, and
e) at the time or after induction adding an amount of methionine to the liquid medium while culturing said host cells to produce the recombinant protein,
wherein the amount of methionine per kg of liquid medium added in step e) is higher than the amount of methionine per kg of liquid medium contained in the liquid medium in step b).

22. A process for reducing norleucine misincorporation during the production a recombinant protein comprising the steps of:
a) providing host cells capable of producing a recombinant protein,
b) providing an amount of liquid medium containing between 0 and 1 g methionine per kg of liquid medium,
c) culturing said host cells in the liquid medium,
d) inducing production of the recombinant protein in the liquid medium, and
e) at the time or after induction adding an amount of methionine to the liquid medium while culturing said host cells to produce the recombinant protein,
wherein the amount of methionine per kg of liquid medium added in step e) is higher than the amount of methionine per kg of liquid medium contained in the liquid medium in step b).

23. The process according to claim 21, wherein the host cells are harvested after step e) and the amount of methionine added in step e) is such that the concentration of methionine in the liquid medium at or immediately prior to harvest is between 0.25 g/kg and 1.5 g/kg.

24. The process according to claim 21, wherein the amount of methionine added in step e) is between 0.25 g and 2.0 g per kg of liquid medium provided in step b).

25. The process according to claim 21, wherein the amount of methionine contained in the liquid medium in step b) and/or step c) is less than 0.5 g/kg.

26. The process according to claim 25, wherein no methionine is contained in the liquid medium in step b) and/or step c).

27. The process according to claim 21, wherein step c) comprises growing the culture to an OD600 of at least 50.

28. The process according to claim 21, wherein step c) and/or step e) comprise a step of culturing said host cells in fed-batch culture.

29. The process according to claim 21, wherein feed containing a carbon source is added during step c) and step e) and the amount of carbon source added per unit time to the liquid medium in step e), is lower than in step c).

30. The process according to claim 21, wherein step d) is initiated when a 50% increase in dissolved oxygen occurs in the liquid medium or when a predefined OD600 is reached.

31. The process according to claim 21, wherein said host cells are bacterial cells.

32. The process according to claim 21, wherein no leucine and/or isoleucine is contained in the liquid medium in step b) and/or step c) and/or step e).

33. The process according to claim 21, wherein said host cells produce recombinant protein upon induction with IPTG and optionally wherein step d) comprises addition of IPTG.

34. The process according to claim 21, wherein the duration of step e) is between 12 and 96 hours or the duration of step e) is between about 12 and about 96 hours.

35. The process according to claim 21, wherein more than 75% of the carbon source consists of glycerol.

36. The process according to claim 21, wherein the recombinant protein is an antibody or a Fab′ fragment.

37. The process according to claim 21, wherein at least step e) is carried out in a bioreactor.

38. The process according to claim 21, wherein the process comprises the step of recovering the recombinant protein, a further step of purifying the recombinant protein and, optionally, a further step of formulating the recombinant protein.

39. The process according to claim 38, wherein the process comprises lyophilizing the recombinant protein.

40. A recombinant protein preparation obtained by the process according to claim 21.
